(19) United States
(12) Patent Application Publication
(10) Pub. No.: US 2024/0285736 A1
(43) Pub. Date: Aug. 29, 2024

(54) Treatment and prevention of pre-eclampsia

(71) Applicant: Pharming Intellectual Property B.V., Leiden (NL)

(72) Inventors: Sijmen DE VRIES, Leiden (NL); Bruno GIANNETTI, Leiden (NL)

(73) Assignee: Pharming Intellectual Property B.V., Leiden (NL)

(21) Appl. No.: 18/655,414

(22) Filed: May 6, 2024

Related U.S. Application Data
(62) Division of application No. 16/975,725, filed on Aug. 26, 2020.

(30) Foreign Application Priority Data
Feb. 28, 2018 (EP) 18159064.7

(51) Int. Cl. A61K 38/57; A61P 9/12

(52) U.S. Cl. CPC A61K 38/57; A61P 9/12

(57) ABSTRACT

The present invention relates to the field of medicine, specifically to the prevention and treatment of pre-eclampsia.

### FIELD OF THE INVENTION

The present invention relates to the field of medicine, specifically to the prevention and treatment of pre-eclampsia.

### BACKGROUND OF THE INVENTION

Pre-eclampsia (PE) is a placental disease [1] characterized by insufficiency of the uteroplacental circulation [2], and which affects 10-12% of all pregnancies and is a major factor in the perinatal mortality rate. Pre-eclampsia is a severe complication of human pregnancy characterized by development of hypertension and proteinuria and it affects maternal and foetal morbidity and mortality worldwide [3]. In developed countries, pre-eclampsia has been reported to complicate 1 to 4% of all pregnancies [3] while in developing countries the prevalence can be as high as 17% [4]. In developing countries hypertensive disorders of pregnancy heavily contribute to all maternal and foetal deaths [5]. There is evidence that one or more placental-derived factors are released into the maternal circulation which either directly or indirectly cause maternal endothelial dysfunction and ensuing maternal problems with activation of the clotting system increased vascular permeability and ischemia in maternal organs secondary to vasoconstriction [6]. It has been postulated that administration of a P-type IPG antagonist could be effective in the treatment of pre-eclampsia (WO9810791). To date however, there is no treatment that is satisfactorily effective for the prevention or treatment of pre-eclampsia except for delivery of the baby.

### SUMMARY OF THE INVENTION

The invention provides for a compound for use in the prevention of pre-eclampsia in a subject at risk of pre-eclampsia or for use in the treatment of a subject suffering from pre-eclampsia, wherein said compound is a complement inhibitor.

The invention further provides for a method of prevention of pre-eclampsia in a subject at risk of pre-eclampsia or a method of treatment of a subject suffering from pre-eclampsia, comprising administration of a complement inhibitor to the subject.

The invention further provides for the use of a complement inhibitor for the prevention of pre-eclampsia in a subject at risk of pre-eclampsia or for the treatment of a subject suffering from pre-eclampsia, comprising administration of a complement inhibitor to the subject.

### DETAILED DESCRIPTION OF THE INVENTION

The inventors arrived at the surprising finding that inhibition of the complement system can be effective for the prevention and treatment of pre-eclampsia.

Accordingly, in a first aspect, the invention provides for a compound for use in the prevention of pre-eclampsia in a subject at risk of pre-eclampsia or for use in the treatment of a subject suffering from pre-eclampsia, wherein said compound is a complement inhibitor. Herein, the compound for use is referred to as the compound according to the invention. In all embodiments of the invention, a complement inhibitor is construed as any compound that is capable of inhibiting the complement pathway to at least some extent, such as one step within the pathway. Pre-eclampsia is construed herein as defined in the background section here above. The compound according to the invention may be any compound, it may be a small molecule, a protein, peptide, an antibody, an enzyme, an enzyme inhibitor such as a protease inhibitor or a chelator. Prevention of pre-eclampsia is herein construed as a delay of the onset of pre-eclampsia and/or a significant reduction of pre-eclampsia when the onset of pre-eclampsia occurs in a person at risk of pre-eclampsia. Treatment of pre-eclampsia is herein construed as a significant reduction of pre-eclampsia in a subject suffering from pre-eclampsia.

In the embodiments of the invention, the compound for use according to the invention, the complement inhibitor, can be: an antibody, an agent inhibiting Factor XII activity, an agent inhibiting Kallikrein activity, a bradykinin receptor antagonist or blocker and/or an agent inhibiting C1 esterase activity. In the embodiments of the invention, the complement inhibitor can be a C1 esterase inhibitor. The C1 esterase inhibitor may be any C1 esterase inhibitor known to the person skilled in the art. In an embodiment, the C1 esterase inhibitor is an antibody directed against (human) C1 inhibitor. In the embodiments of the invention, the C1 esterase inhibitor can be a plasma-derived C1 esterase inhibitor. In the embodiments of the invention, the C1 esterase inhibitor can be a recombinant C1 esterase inhibitor, preferably a C1 esterase inhibitor having an amino acid sequence that is substantially identical to the amino acid sequence of human plasma-derived C1 esterase inhibitor. The recombinant C1 esterase inhibitor can be any recombinant C1 esterase inhibitor known the person skilled in the art. It may be produced recombinantly in microbial cells, such as tissue culture cells. The tissue culture cell can be a mammalian tissue culture cell, such as a Chinese Hamster Ovarian (CHO) cell or a human tissue culture cell (see e.g. WO2016/081889, which is herein incorporated by reference). The recombinant C1 esterase inhibitor can be produced in transgenic animals, such as in a transgenic non-human mammal, preferably a mouse, goat, bovine, sheep, porcine or an animal from the order Lagomorpha, such as a Leporadae, including a rabbit. In an embodiment, the recombinant C1 esterase inhibitor is one produced according to the methods in WO01/57079, which is herein incorporated by reference.

In the embodiments of the invention, the C1 esterase inhibitor can be a modified C1 esterase inhibitor as compared to human plasma-derived C1 esterase inhibitor. It can be modified to modulate the plasma half-life of the C1 esterase inhibitor. A specific modified C1 esterase inhibitor is conjugated to enhance the plasma half-life. An exemplary conjugated C1 esterase inhibitor to enhance half-life is a conjugated C1 esterase inhibitor according to WO2017/176798, which is herein incorporated by reference, such as a polysialic acid (PSA)-conjugated C1 esterase inhibitor, more preferably a polyethylene glycol (PEG)-conjugated C1 esterase inhibitor. The modification of the C1 esterase inhibitor can be a modified carbohydrate structure as compared to human plasma-derived C1 esterase inhibitor. A specific modified C1 esterase inhibitor has a reduced level of terminal sialic acid residues as compared to plasma derived C1 esterase inhibitor, wherein said reduced level of terminal sialic acid residues may result in a reduction of plasma half-life to less than 6 hours. A specific C1 esterase inhibitor having a reduced level of terminal sialic acid residues as compared to plasma derived C1 esterase inhibitor is a C1 esterase inhibitor according to WO01/57079, WO2004/100982 and WO2007/073186 which are herein incorporated by reference. The compound according to the invention can be administered as such and can be administered comprised in a pharmaceutical composition. The pharmaceutical composition can comprise a pharmaceutically accepted excipient and/or can comprise a further pharmaceutical compound. The compound according to the invention may be administered by any means known to the person skilled in the art, such as but not limited, to intravenous, transdermal and subcutaneous administration. Intravenous administration is extensively described in WO01/57079, WO2004/100982 and WO2007/073186. Subcutaneous administration is preferably performed as in WO2014/145519, U.S. Pat. No. 9,616,111B2 and EP2968434B1, which are herein incorporated by reference. In the embodiments of the invention, the compound according to the invention can be administered to the subject at least once a month, or at least once a week. The compound according to the invention can be administered at least once, twice, three or four times a month, at least once, twice, three, four, five, six or seven times a week or can be administered, every other day, daily, or twice a day.

When the compound according to the invention is a C1 esterase inhibitor, the compound can be administered in a dose ranging from 25 units/kg body weight to 100 units/kg body weight per administration, preferably ranging from 50 units/kg body weight to 100 units/kg body weight per administration. Per administration the dose can be 25 units/kg body weight, 50 units/kg body weight, 100 units/kg body weight. The total dose per administration can be 1000 units, 1400 units, 1500 units, 2000 units, 2100 units, 2800 units, 3000 units, 3500 units, 4000 units, 4200 units, 4500 units, 4900 units, 5000 units, 5600 units, 6000 units, 6300 units, 7000 units, 7500 units, 8000 units, 8400 units or 9000 units C1 inhibitor.

In the embodiments of the invention, the subject can be a pregnant mammal, preferably a pregnant human.

In the embodiments of the invention, the subject suffering from pre-eclampsia can be suffering from early-onset pre-eclampsia (<34 weeks gestational age) or from late-onset pre-eclampsia (>34 weeks gestational age).

The diagnosis of pre-eclampsia or of a risk of pre-eclampsia can be made by any means and assay known to the person skilled in the art. The diagnosis may e.g. be made by assessing whether there is occurrence of hypertension en proteinuria. The diagnosis can be made by measurement of P-type inositolphosphoglycans (P-type IPG) in a bodily fluid, such as in blood or urine.

Accordingly, the invention provides for a compound for use according to the invention, wherein the subject is diagnosed with pre-eclampsia or being at risk of pre-eclampsia by measurement of P-type inositolphosphoglycans (P-type IPG) in a bodily fluid, such as in blood or urine. In an embodiment, the invention provides for a compound for use according to the invention, wherein the subject is diagnosed with of being at risk of pre-eclampsia by measurement of P-type inositolphosphoglycans (P-type IPG) in a bodily fluid, such as in blood or urine. Preferably, the P-type inositolphosphoglycans (P-type IPG) is placenta-derived P-type inositolphosphoglycans (P-type IPG). In the embodiments of the invention, the measurement can be performed by any means known to the person skilled in the art, such as by using the method as described in WO9810791, which is herein incorporated by reference. Preferably, the measurement is performed according to experiments A-1, A-2, A-5 and A-6 of WO9810791. For reference, the here mentioned parts of WO9810791 are copied here below.

The activity of P-and A-type IPGs in urine and placental extracts were studied using specific bioassay procedures. IPG P-type was determined using the activation of PDH phosphatase [7]. The PDH complex and PDH phosphatase (metal-dependent form) were prepared from beef heart as described by Lilley et al. [7] and the assay of the activation of the phosphatase was performed by the spectrophotometric variant of the two-stage system described by these authors. This assay is considered to be a characteristic feature of IPG P-type (see Lamer et al. [8]). IPG A-type was determined by the stimulation of lipogenesis as measured by the incorporation of [U¹⁴C] glucose into the lipids of adipocytes isolated from epididymal fat pads by the method of Rodbell [9]. A high degree of specificity for IPG A-type was found for this bioassay.

A straight line relationship between added IPGs and the stimulation of PDH phosphatase activity (IPG P-type) and lipogenesis in intact adipocytes (IPG A-type) was obtained; this relationship held at least up to a stimulation of −1-250%. These observations provided a basis for a unit to be defined and used for the purpose of comparison of yields of IPGs from different tissues and urine samples. Linearity between IPG added and the percentage change in response, has been observed by others (see Lilley et al. [7] and Newman et al. [10]), although Asplin et al. [11] did not show linearity in their study on IPGs in human urine from normal and diabetic subjects, an effect which was particularly marked with the IPG A-type (pH 1.3 fraction).

Extraction of IPG P-type and IPG-A type from urine was performed as described by Asplin et. al. [11]. The final fractions were freeze dried and stored at −20° C. For use, the IPG fractions were resuspended in water, immediately before assay, so that 10 μl of redissolved IPG corresponded to 10 ml urine.

In view of the possibility that high, and varying, amounts of IPGs might be excreted in the different groups of pregnant and pre-eclamptic subjects, and in order to ensure that the capacity of the resin was well in excess of the load applied, preliminary test runs were made to determine the optimal ratio of resin to starting urine volume. Linearity of recovery was obtained up to 100 ml urine per 18 g resin. In the present study, the ratio of 30 ml urine to 18 g resin was maintained to allow for variation in IPG content.

Expression of results: A unit of IPG is defined as the amount causing a 50% activation in the basal level of the test system.

The yield of IPGS in urine is given on three different bases:
(i) Percentage stimulation of the test system by 10 μl final urine extract (Col 1), allowing direct comparison with data of Asplin et. al. [11]
(ii) Units of IPG per 1 mmol creatinine.
(iii) Units of IPG found in a sample of a 24 hour collection or urine; i.e.: the total daily output at that stage of gestation.

In a second aspect, the invention provides for a method of prevention of pre-eclampsia in a subject at risk of pre-eclampsia and a method of treatment of a subject suffering from pre-eclampsia, comprising administration of a complement inhibitor to the subject. The features of this aspect of the invention can be those of the first aspect of the invention.

In this aspect of the invention, the subject can be diagnosed with pre-eclampsia or being at risk of pre-eclampsia by measurement of placenta-derived P-type inositolphosphoglycans (P-type IPG) in a bodily fluid, such as in blood or urine. In the embodiments of the invention, the measurement can be performed using the method as described in WO9810791, which is herein incorporated by reference. Preferably, the measurement is performed according to experiments A-1, A-2, A-5 and A-6 of WO9810791.

In a third aspect, the invention provides for the use of a complement inhibitor for the prevention of pre-eclampsia in a subject at risk of pre-eclampsia and for the treatment of a subject suffering from pre-eclampsia, comprising administration of a complement inhibitor to the subject. The features of this aspect of the invention can be those of the first and second aspect of the invention. In this aspect of the invention, the subject can be diagnosed with pre-eclampsia or being at risk of pre-eclampsia by measurement of placenta-derived P-type inositolphosphoglycans (P-type IPG) in a bodily fluid, such as in blood or urine. In the embodiments of the invention, the measurement can be performed using the method as described in WO9810791, which is herein incorporated by reference. Preferably, the measurement is performed according to experiments A-1, A-2, A-5 and A-6 of WO9810791.

In a fourth aspect, the invention provides for a complement inhibitor for the manufacture of a medicament for the prevention of pre-eclampsia in a subject at risk of pre-eclampsia and for the treatment of a subject suffering from pre-eclampsia, comprising administration of the complement inhibitor to the subject. The features of this aspect of the invention can be those of the first, second and third aspect of the invention. In this aspect of the invention, the subject can be diagnosed with pre-eclampsia or being at risk of pre-eclampsia by measurement of placenta-derived P-type inositolphosphoglycans (P-type IPG) in a bodily fluid, such as in blood or urine. In the embodiments of the invention, the measurement can be performed using the method as described in WO9810791, which is herein incorporated by reference. Preferably, the measurement is performed according to experiments A-1, A-2, A-5 and A-6 of WO9810791.

Unless otherwise indicated each embodiment as described herein may be combined with another embodiment as described herein.

### Definitions

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

Herein, one unit (U) of C1 esterase inhibitor is the amount of C1 esterase inhibitor present in 1 milliliter of human plasma. One such unit corresponds to approximately 275 microgram plasma-derived C1 esterase inhibitor.

All patent and literature references cited in the present specification are hereby incorporated by reference in their entirety.

### Embodiments of the Invention

1. A compound for use in the prevention of pre-eclampsia in a subject at risk of pre-eclampsia or for use in the treatment of a subject suffering from pre-eclampsia, wherein said compound is a complement inhibitor.
2. A compound for use according to embodiment 1, wherein the complement inhibitor is: an antibody, an agent inhibiting Factor XII activity, an agent inhibiting Kallikrein activity, an agent inhibiting bradykinin activity and/or an agent inhibiting C1 esterase activity.
3. A compound for use according to embodiment 1 or 2, wherein the complement inhibitor is a C1 esterase inhibitor.
4. A compound for use according to embodiment 4, wherein the C1 esterase inhibitor is an antibody.
5. A compound for use according to embodiment 4, wherein the C1 esterase inhibitor is a plasma-derived C1 esterase inhibitor.
6. A compound for use according to embodiment 4, wherein the C1 esterase inhibitor is a recombinant C1 esterase inhibitor, such as a C1 esterase inhibitor having an amino acid sequence that is substantially identical to the amino acid sequence of human plasma-derived C1 esterase inhibitor.
7. A compound for use according to embodiment 4, wherein the recombinant esterase inhibitor is produced in a transgenic non-human mammal, such as a mouse, goat, bovine, sheep, porcine or an animal from the order Lagomorpha, such as a Leporadae, including a rabbit.
8. A compound for use according to any one of embodiments 5-7, wherein the C1 esterase inhibitor has a modified carbohydrate structure as compared to human plasma-derived C1 esterase inhibitor.
9. A compound for use according to embodiment 8, wherein the C1 esterase inhibitor has a reduced level of terminal sialic acid residues as compared to plasma derived C1 inhibitor, wherein said reduced level of terminal sialic acid residues preferably results in a plasma half-life of less than 6 hours.
10. A compound for use according to any one of the preceding embodiments, wherein the compound is administered to the subject at least once a month, or at least once a week.
11. A compound for use according to any one of embodiments 5-10, wherein the compound is administered in a dose ranging from 25 units/kg body weight to 100 units/kg body weight per administration, or ranging from 50 units/kg body weight to 100 units/kg body weight per administration.
12. A compound for use according to any one of the preceding embodiments, wherein the subject is a pregnant mammal, preferably a pregnant human.
13. A compound for use according to any of the preceding embodiments, wherein the subject suffering from pre-eclampsia is suffering from early-onset pre-eclampsia (<34 weeks gestational age) or from late-onset pre-eclampsia (>34 weeks gestational age).
14. A compound for use according to any of the preceding embodiments, wherein the subject is diagnosed with pre-eclampsia or being at risk of pre-eclampsia by measurement of P-type inositolphosphoglycans (P-type IPG) in a bodily fluid, preferably in blood or urine.
15. A compound for use according to any of the preceding embodiments, wherein the subject is diagnosed with pre-eclampsia or being at risk of pre-eclampsia by measurement of placenta-derived P-type inositolphosphoglycans (P-type IPG) in a bodily fluid, preferably in blood or urine.
16. A compound for use according to embodiment 14 or 15, wherein the measurement is performed using the method as described in WO9810791.
17. A method of prevention of pre-eclampsia in a subject at risk of pre-eclampsia or a method of treatment of a subject suffering from pre-eclampsia, comprising administration of a complement inhibitor to the subject.
18. A method of prevention of pre-eclampsia in a subject at risk of pre-eclampsia or a method of treatment of a subject suffering from pre-eclampsia according to embodiment 17, wherein the subject is diagnosed with pre-eclampsia or being at risk of pre-eclampsia by measurement of placenta-derived P-type inositolphosphoglycans (P-type IPG) in a bodily fluid, such as in blood or urine.
19. A method of prevention of pre-eclampsia in a subject at risk of pre-eclampsia or a method of treatment of a subject suffering from pre-eclampsia according to embodiment 17 or 18, wherein the measurement is performed using the method as described in WO9810791.
20. Use of a complement inhibitor for the prevention of pre-eclampsia in a subject at risk of pre-eclampsia or for the treatment of a subject suffering from pre-eclampsia, comprising administration of a complement inhibitor to the subject.
21. Use according to embodiment 20, wherein the subject is diagnosed with pre-eclampsia or being at risk of pre-eclampsia by measurement of placenta-derived P-type inositolphosphoglycans (P-type IPG) in a bodily fluid, such as in blood or urine.
22. Use according to embodiment 20 or 21, wherein the measurement is performed using the method as described in WO9810791.

The following examples are offered for illustrative purposes only, and are not intended to limit the scope of the present invention in any way.

### EXAMPLES

### Example 1

C1 esterase inhibitor from transgenic rabbits prepared as in WO01/57079 (Ruconest™, Pharming, the Netherlands) is administered on daily basis in a 50 units/kg bodyweight dose to pregnant human subjects suffering from early-onset pre-eclampsia (<34 weeks gestational age) or from late-onset pre-eclampsia (>34 weeks gestational age). A control group receives no treatment except for the state of the art hospitalization. On average, the treated groups demonstrate significant positive effects of the treatment as demonstrated by lower blood pressure and lower proteinuria.

### Example 2

C1 esterase inhibitor from transgenic rabbits prepared as in WO01/57079 (Ruconest™, Pharming, the Netherlands) is administered on daily basis in a 50 units/kg bodyweight dose to pregnant human subjects at risk of pre-eclampsia (>34 weeks gestational age). A control group receives no treatment except for the state of the art hospitalization. On average, the treated group demonstrates significant positive effects of the treatment as demonstrated by no or later onset of pre-eclampsia and lower blood pressure and lower proteinuria when pre-eclampsia does occur.

### Example 3

### Synopsis Pre-Eclampsia Clinical Study

Study Title: A Phase I/II, Open Label, Proof of Concept Study to investigate Tolerability and Safety of Treatment with Recombinant Human C1 Inhibitor (conestat alfa) in Patients with pre-eclampsia.

Study Phase: Phase I/II, proof of concept study

Number of Patients: Up to 30 patients can be enrolled. Recruitment will stop once 20 patients completed the treatment period or 30 patients have been enrolled whichever comes first. Already enrolled patients will complete the study as per protocol.

### Rationale

The current trial intends to evaluate the tolerability and safety of recombinant human C1 esterase inhibitor (rhC1INH) conestat alfa—in the patient with pre-eclampsia and further explore the efficacy of such a treatment.

### Objectives

### Primary

To evaluate the tolerability and safety of the treatment with rhC1INH (conestat alfa) on top of Standard Care, for patients with pre-eclampsia.

### Secondary

To evaluate the efficacy of treatment with rhC1INH (conestat alfa) on top of Standard Care, for patients with pre-eclampsia.

### Exploratory

To further characterize the clinical safety of treatment with rhC1INH (conestat alfa) on top of Standard Care, for patients with pre-eclampsia by measuring evolution of laboratory parameters like platelet count, LDH, ALAT, ASAT, Kreat, Hb, Ht, urine protein, urine kreatinine,

To identify changes in biomarkers of pre-eclampsia (PIGF, VEGF, sFlt-1, IPG, podocytes level) following rhC1INH (conestat alfa) treatment.

To analyze complement activation and other immune characteristics in the mother's blood and urine (C4, C1q, C5b and factor H) and in the placenta and cord blood (C4, C1q, C5b and factor H, leucocyte count and FACS analysis)

To evaluate immune system in the baby's blood after birth by measuring complement activation (C4, C1q, C5b, factor H) leucocyte count and performing a FACS analysis.

To evaluate plasma pharmacokinetic profile of rhC1INH (conestat alfa) in pregnant women.

### Study Design:

This is an open label, proof of concept study on the treatment with rhC1INH (conestat alfa) of patients with pre-eclampsia between 27-34 weeks gestation. The study will consist of a screening visit, an enrolment visit, a treatment period and a follow-up period.

### Study Population

Up to 30 pregnant women between 27-34 weeks of gestation referred to hospital care centers for management of their pregnancy with pre-eclampsia, fulfilling the inclusion criteria, are eligible for participation in the study:

### Intervention

Twice weekly open label intravenous treatment with conestat alfa at a dose of 50 units/kg (based on body weight at start of treatment) up to a maximum of 4200 units on top of Standard Care.

### Main Study Parameters/Endpoints

### Primary Endpoint

Incidence and severity of adverse events
Number and percentage of patients who discontinue investigational product or withdraw from the study

### Secondary Endpoints

Time from start of conestat alfa to day of delivery
Proportion of patients reaching gestation week 37

### Explorative Endpoints

### Mother

Biochemistry and hematology parameters over time, including safety parameters like platelet count, LDH, ALAT, ASAT, creatinine, Hb, Ht.
Urine protein and creatinine level
Number of women who develop HELLP syndrome, eclampsia.
Number of women with placental abruption
Plasma concentration of C1INH—pharmacokinetic profile analysis
Biomarkers in blood sFlt-1, VEGF and PIGF
Biomarkers in urine: IPG and podocyte levels
Gestational age at birth
Incidence of admission to a neonatal intensive care unit as applicable
Time spent in the neonatal intensive care unit
Normalization of uterine Doppler flow profiles
Normalization of Doppler profiles of the Umbilical artery and/or medial cerebral artery
Quantification of complement activation in placental tissue (C1q)
Quantification of complement activation in maternal blood and urine samples (C4, C1q, C5b, factor H)
Proportion of patients reaching gestation week 34, or 30

### Baby

Birthweight
Delivery of healthy baby, defined as: a term baby, normal weight (=per country/part of the world), normal APGAR score, no congenital abnormalities, normal head circumference
Incidence of neonatal:
   Necrotizing enterocolitis
   Respiratory distress syndrome,
   Cerebral hemorrhage,
   Grade 3-4 intraventricular hemorrhage,
   Bronchopulmonary dysplasia
Normal Immune system in the baby's blood and cord blood by measuring complement activation (C4, C1q, C5b, factor H), leucocyte count and a performing a FACS analysis
Normal pediatric echocardiography after delivery

### Efficacy Analyses

Efficacy analyses will be performed for the following:
Time from start of treatment to delivery
Patients reaching gestation week 37

### Pharmacokinetic Profile Analyses

The mean plasma concentrations of C1INH over time will be plotted and analyzed visually.

### Biomarkers for PE

Biomarkers for pre-eclampsia (sFlt-1, PIGF, VEGF, podocytes levels and IPG)
Quantification of complement activation in placental tissue and cord blood (C4, C1q, C5b, factor H)
Quantification of complement activation in maternal blood and urine samples (C4, C1q, C5b, factor H)
Quantification of complement activation in baby's blood (C4, C1q, C5b, factor H) at 3 months follow up visit

### REFERENCES

1. Redman, C.W.G., 1991. Pre-eclampsia and the placenta. Placenta, 12(4), pp. 301-308.
2. Robertson, W.B., Brosens, I. and Dixon, H.G., 1967. The pathological response of the vessels of the placental bed to hypertensive pregnancy. The Journal of Pathology, 93(2), pp. 581-592.
3. Steegers, E.A., von Dadelszen, P., Duvekot, J.J. and Pijnenborg, R., 2010. Pre-eclampsia. The Lancet, 376(9741), pp. 631-644.
4. Osungbade, K.O. and Ige, O.K., 2011. Public health perspectives of preeclampsia in developing countries: implication for health system strengthening. Journal of pregnancy, 2011.
5. The world health report 2005—make every mother and child count. WHO, Geneva. Available at: www.who.int/whr/2005/en/.
6. Romero, G., 1991. Inositolglycans and cellular signalling. Cell biology international reports, 15(9), pp. 827-852.
7. Lilley, K., Zhang, C., Villar-Palasi, C., Larner, J. and Huang, L., 1992. Insulin mediator stimulation of pyruvate dehydrogenase phosphatases. Archives of biochemistry and biophysics, 296(1), pp. 170-174.
8. Larner, J., Huang, L.C., Suzuki, S., Tang, G., Zhang, C., Schwartz, C.F.W., Romero, G., Luttrell, L. and Kennington, A.S., 1989. Insulin mediators and the control of pyruvate dehydrogenase complex. Annals of the New York Academy of Sciences, 573(1), pp. 297-305.
9. Rodbell, M., 1964. The metabolism of isolated fat cells. Comprehensive Physiology.
10. Newman, J., Armstrong, J.M. and Bornstein, J., 1985. Assay of insulin mediator activity with soluble pyruvate dehydrogenase phosphatase. Endocrinology, 116(5), pp. 1912-1919.
11. Asplin, I., Galasko, G. and Larner, J., 1993. Chiro-inositol deficiency and insulin resistance: a comparison of the chiro-inositol-and the myo-inositol-containing insulin mediators isolated from urine, hemodialysate, and muscle of control and type II diabetic subjects. Proceedings of the National Academy of Sciences, 90(13), pp. 5924-5928.

1. A method treatment for the prevention of pre-eclampsia in a subject at risk of pre-eclampsia comprising administration of a C1 esterase inhibitor to the subject.

2. The method of treatment according to claim, wherein the C1 esterase inhibitor is a plasma-derived C1 esterase inhibitor.

3. The method of treatment according to claim 1, wherein the C1 esterase inhibitor is a recombinant C1 esterase inhibitor.

4. The method of treatment according to claim 1, wherein the recombinant esterase inhibitor is produced in a transgenic non-human mammal.

5. The method of treatment according to claim 1, wherein the C1 esterase inhibitor has a modified carbohydrate structure as compared to human plasma-derived C1 esterase inhibitor.

6. The method of treatment according to claim 1, wherein the C1 esterase inhibitor has a reduced level of terminal sialic acid residues as compared to plasma derived C1 inhibitor.

7. The method of treatment according to claim 1, wherein the compound is administered to the subject at least once a month, or at least once a week.

8. The method of treatment according to claim 1, wherein the compound is administered in a dose ranging from 25 units/kg body weight to 100 units/kg body weight per administration, or ranging from 50 units/kg body weight to 100 units/kg body weight per administration.

9. The method of treatment according to claim 1, wherein the subject is a pregnant mammal.

10. The method of treatment according to claim 1, wherein the subject suffering from pre-eclampsia is suffering from early-onset pre-eclampsia (<34 weeks gestational age) or from late-onset pre-eclampsia (>34 weeks gestational age).

11. The method of treatment according to claim 1, wherein the subject is diagnosed of being at risk of pre-eclampsia by measurement of P-type inositolphosphoglycans (P-type IPG) in a bodily fluid.

12. The method of treatment according to claim 1, wherein the subject is diagnosed of being at risk of pre-eclampsia by measurement of placenta-derived P-type inositolphosphoglycans (P-type IPG) in a bodily fluid.

13. The method of treatment according to claim 1, wherein the measurement is performed using the method as described in examples A-1, A-2, A-5 and A-6 of WO9810791.

14. The method of treatment according to claim 11, wherein the transgenic non-human mammal is a mouse, goat, bovine, sheep, porcine or an animal from the order Lagomorpha.

15. The method of treatment according to claim 14, wherein the transgenic non-human mammal is a Leporadae, including a rabbit.

16. The method of treatment according to claim 6, wherein said reduced level of terminal sialic acid residues results in a plasma half-life of less than 6 hours.

17. The method of treatment according to claim 9, wherein the pregnant mammal is a pregnant human.

18. The method of treatment according to claim 11, wherein the bodily fluid is blood or urine.

19. The method of treatment according to claim 12, wherein the bodily fluid is blood or urine.
